(19) United States
(12) Patent Application Publication
(10) Pub. No.: US 2020/0206023 A1
(43) Pub. Date: Jul. 2, 2020

(54) OPHTHALMIC DEVICE FOR TREATING DRY EYE

(71) Applicant: Verily Life Sciences LLC, South San Francisco, CA (US)

(72) Inventors: Anupam Pathak, Los Altos Hills, CA (US); Dimitri Azar, San Francisco, CA (US); Todd Whitehurst, Belmont, CA (US)

(21) Appl. No.: 16/696,965

(22) Filed: Nov. 26, 2019

Related U.S. Application Data
(60) Provisional application No. 62/786,171, filed on Dec. 28, 2018.

(51) Int. Cl. A61F 7/00; A61F 7/03

(52) U.S. Cl. CPC A61F 7/007; A61F 7/03; A61F 7/0085; A61F 2007/0004; A61F 2007/0054; A61F 2007/0096; A61F 2007/0075; A61F 2007/0094; A61F 2007/0056; A61F 2007/0086

(57) ABSTRACT

Ophthalmic devices and systems for treating dry eye are described. An ophthalmic device includes a housing having a convex anterior surface and a concave posterior surface and a thermal regulator disposed therein. In an example, the thermal regulator includes a heating element positioned to heat the convex anterior surface. In an example, the thermal regulator includes a cooling element positioned to cool the concave posterior surface. The convex anterior surface is shaped to contact a portion of conjunctiva of an eye adjacent to Meibomian glands of the eye, such as the palpebral conjunctiva, when the ophthalmic device is mounted to the eye. The concave posterior surface is shaped to contact a portion of the conjunctiva adjacent to scleral nerves of the eye, such as the bulbar conjunctiva, when the ophthalmic device is mounted to the eye.

### CROSS-REFERENCE TO RELATED APPLICATION

This application claims the benefit of U.S. Provisional Application No. 62/786,171, filed Dec. 28, 2018, which is hereby incorporated by reference in its entirety.

### TECHNICAL FIELD

This disclosure relates generally to ophthalmic devices for treating dry eye, and, in particular but not exclusively, relates to ophthalmic devices for treating dry eye through heating Meibomian glands.

### BACKGROUND INFORMATION

It is estimated that about 16 million people have chronic dry eye syndrome in the U.S. alone. About 60-70% of this population is experiencing chronic dry eye due to a dysfunction of the Meibomian gland in supplying meibum, an oily substance, to the eye. This gland is located at the rim of the eyelids, and is responsible for the supply of meibum, which prevents the evaporation of the eye's tear film. Conventional treatment advises patients to apply warm compresses to the eye in order to stimulate the release of additional meibum.

### BRIEF DESCRIPTION OF THE DRAWINGS

Non-limiting and non-exhaustive embodiments of the invention are described with reference to the following figures, wherein like reference numerals refer to like parts throughout the various views unless otherwise specified. Not all instances of an element are necessarily labeled so as not to clutter the drawings where appropriate. The drawings are not necessarily to scale, emphasis instead being placed upon illustrating the principles being described.

FIG. 1A is a top-down plan view of an ophthalmic device, in accordance with an embodiment of the disclosure.

FIG. 1B is a cross-sectional view of the ophthalmic device of FIG. 1A.

FIG. 1C is another cross-sectional view of the ophthalmic device of FIG. 1A shown mounted under an eyelid of an eye, in accordance with an embodiment of the disclosure.

FIG. 2A is a top-down plan view of an ophthalmic device, in accordance with an embodiment of the disclosure.

FIG. 2B is a bottom-up plan view of the ophthalmic device of FIG. 2A.

FIG. 2C is a cross-sectional view of the ophthalmic device of FIG. 2A shown mounted to a corneal surface of an eye, in accordance with an embodiment of the disclosure.

FIG. 3A is a cross-sectional view of an ophthalmic device, in accordance with an embodiment of the disclosure.

FIG. 3B is a cross-sectional view of a portion of the ophthalmic device of FIG. 3A.

FIG. 4 is a perspective view of a system, in accordance with an embodiment of the disclosure.

### DETAILED DESCRIPTION

Embodiments of an ophthalmic device and a system for treatment of dry eye are described herein. In the following description numerous specific details are set forth to provide a thorough understanding of the embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

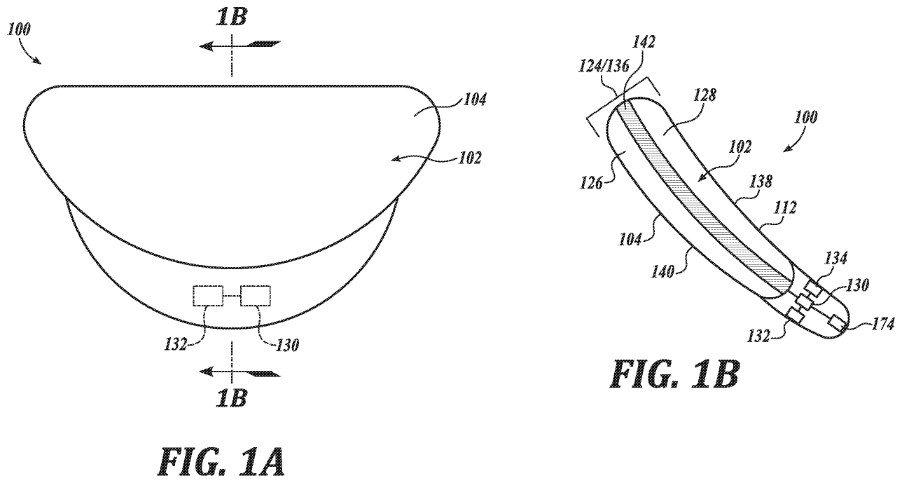

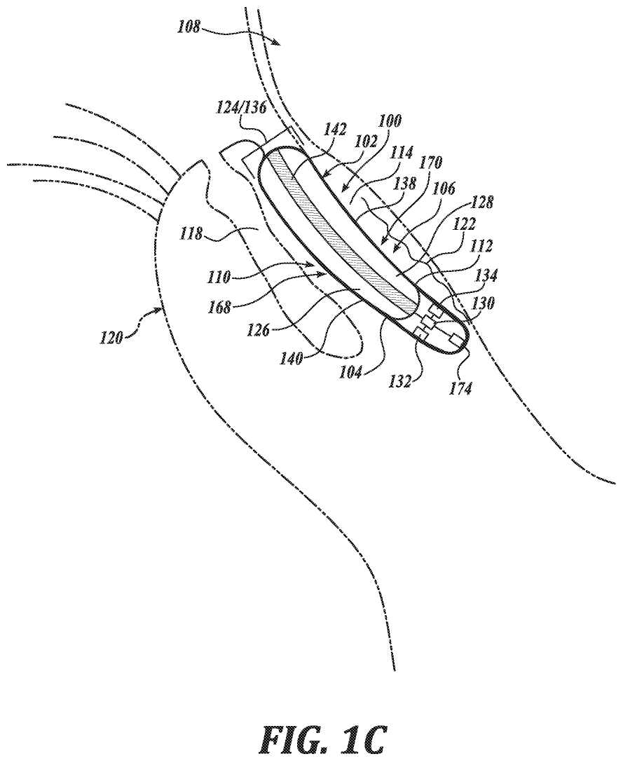

Turning to FIGS. 1A-1C, an ophthalmic device 100, in accordance with an embodiment of the disclosure, will now be described. FIG. 1A is a top-down plan view of the ophthalmic device 100. FIG. B is a cross-sectional view of the ophthalmic device 100. FIG. 1C is another cross-sectional view of the ophthalmic device 100 shown mounted under an eyelid 120 of an eye 108, in accordance with an embodiment of the disclosure.

The ophthalmic device 100 includes a housing 102 and a thermal regulator 124, shown here disposed within the housing 102. The housing 102 defines a convex anterior surface 104 and a concave posterior surface 112. As illustrated in FIG. 1C, the ophthalmic device 100 is shaped to be mounted under an eyelid 120 of the eye 108, such as to a conjunctival surface. As shown, the convex anterior surface 104 is shaped to contact a portion 110 of conjunctiva 106 of the eye 108 adjacent to Meibomian glands 118 of the eye 108 when the ophthalmic device 100 is mounted under an eyelid 120 of the eye 108. Likewise, the concave posterior surface 112 is shaped to contact a second portion 114 of the conjunctiva 106 adjacent to scleral nerves 122 of the eye 108 when the ophthalmic device 100 is mounted under the eyelid 120.

In FIGS. 1A-1C, the ophthalmic device 100 is shown to be an underlid ophthalmic device 100 shaped to be mounted under an eyelid 120 of the eye 108. In this regard, the ophthalmic device 100 is configured to heat Meibomian glands 118 of the eye 108 and/or cool scleral nerves 122 of the eye 108, such as without impeding vision of the eye 108 or being visible outside of the eyelid 120. While an underlid ophthalmic device 100 is illustrated in FIGS. 1A-1C, it will be understood that ophthalmic devices of the present disclosure include other shapes and configurations, such as contact lenses, implanted ophthalmic devices, and the like, as discussed further herein.

As above, the ophthalmic device 100 includes a thermal regulator 124. In the illustrated embodiment, the thermal regulator 124 includes a heating element 126 positioned to heat the convex anterior surface 104. By heating the convex anterior surface 104, the ophthalmic device 100 is configured to heat Meibomian glands 118 of the eye 108 when the ophthalmic device 100 is mounted under the eyelid 120, as shown in FIG. 1C. By heating the Meibomian glands 118, meibum disposed in the Meibomian glands 118 is liquefied or otherwise loosened in the Meibomian glands 118 and exuded from the Meibomian glands 118. As discussed further herein, meibum limits evaporation of tear solutions on the eye 108. By assisting in expressing meibum from the Meibomian glands 118 onto the eye 108, the ophthalmic device 100 is configured to treat dry eye, particularly for those suffering from a dysfunction of the Meibomian glands 118.

The thermal regulator 124 is further shown to include a cooling element 128 positioned to cool the concave posterior surface 112. By cooling the concave posterior surface 112 of the housing 102, the ophthalmic device 100 is configured to cool scleral nerves 122 of the eye 108 when the ophthalmic device 100 is mounted under the eyelid 120 of the eye 108, as shown in FIG. 1C. Cooling scleral nerves 122 can have the effect of inducing lachrymation. Inducing tear generation can also treat dry eye by replacing evaporated tear solution.

In the illustrated embodiment, the thermal regulator 124 comprises the heating element 126 and the cooling element 128. However, it will be understood that, in one embodiment, the thermal regulator 124 includes only the heating element 126. Correspondingly, in one embodiment, the ophthalmic device 100 includes only the cooling element 128. As above, all such embodiments the ophthalmic device 100 are configured to treat one or more causes of dry eye.

The illustrated ophthalmic device 100 is shown to include a thermal insulator 142 disposed between the heating element 126 and the cooling element 128 to thermally insulate the heating element 126 from the cooling element 128. Such a thermal insulator 142 is configured to limit or prevent heat flow between the heating element 126 and the cooling element 128 and provide more efficient heating of the Meibomian glands 118 and cooling of the scleral nerves 122, respectively.

In an embodiment, the thermal regulator 124 includes a Peltier heat pump 136 comprising the heating element 126 and the cooling element 128. In this regard, the heating element 126 comprises a hot side 140 of the Peltier heat pump 136 positioned to heat the convex anterior surface 104. Correspondingly, the cooling element 128 comprises a cold side 138 of the Peltier heat pump 136 positioned to cool the concave posterior surface 112. By powering the Peltier heat pump 136, such as with the power source 174, the Meibomian glands 118 are heated and the scleral nerves 122 are cooled when the ophthalmic device 100 is mounted under the eyelid 120 of the eye 108, as shown in FIG. 1C.

Ophthalmic device 100 is shown to include a controller 130 operatively coupled to the heating element 126 and the cooling element 128. The controller 130 includes logic that, when executed by the controller 130, causes the ophthalmic device 100 to perform operations. Such operations can include heating the convex anterior surface 104 with the heating element 126 and/or cooling the concave posterior surface 112 with the cooling element 128.

The ophthalmic device 100 includes a temperature sensor 132 configured to generate a temperature signal based on an ambient temperature adjacent to the temperature sensor 132. As shown, the temperature sensor 132 is disposed adjacent to the convex anterior surface 104 of the housing 102. Accordingly, the temperature sensor 132 is positioned to contact a portion 110 of the conjunctiva 106, such as the palpebral conjunctiva 168, adjacent to the Meibomian glands 118. In this regard, the temperature sensor 132 is configured to generate a temperature signal indicative of a temperature of the Meibomian glands 118. In an embodiment, the controller 130 includes logic that, when executed by the controller 130, causes the ophthalmic device 100 to perform operations including heating the convex anterior surface 104 with the heating element 126 based upon the temperature signal. In this regard, heat may be applied to the Meibomian glands 118 taking into account a temperature of the Meibomian glands 118 such that, for example, the Meibomian glands 118 are heated sufficiently to express meibum therefrom without overheating the Meibomian glands 118.

The ophthalmic device 100 further includes an osmolality sensor 134 configured to generate an osmolality signal based on an osmolality of a tear solution of the eye 108. As shown, the osmolality sensor 134 is positioned to contact a portion 114 of conjunctiva 106, such as bulbar conjunctiva 170, adjacent to scleral nerves 122 of the eye 108. In this regard, the osmolality sensor 134 is configured to generate an osmolality signal based on an osmolality of a tear solution of the eye 108 when the ophthalmic device 100 is mounted under the eyelid 120, as shown in FIG. 1C. In an embodiment, the osmolality sensor 134 is also configured to generate a moisture signal indicative of an amount of moisture in contact with the osmolality sensor 134, such as an amount of a tear solution in contact with the osmolality sensor 134. Accordingly, in an embodiment, the controller 130 includes logic that, when executed by the controller 130, causes the ophthalmic device 100 to perform operations including cooling the concave posterior surface 112 with the cooling element 128 based upon the osmolality signal and/or moisture signal. In this regard, cooling may be applied to the scleral nerves 122 taking into account an osmolality and/or a volume of a tear solution of the eye 108 such that, for example, the scleral nerves 122 are cooled sufficiently to induce tear production without overcooling the sclera of the eye 108 or generating an excess of tear solution.

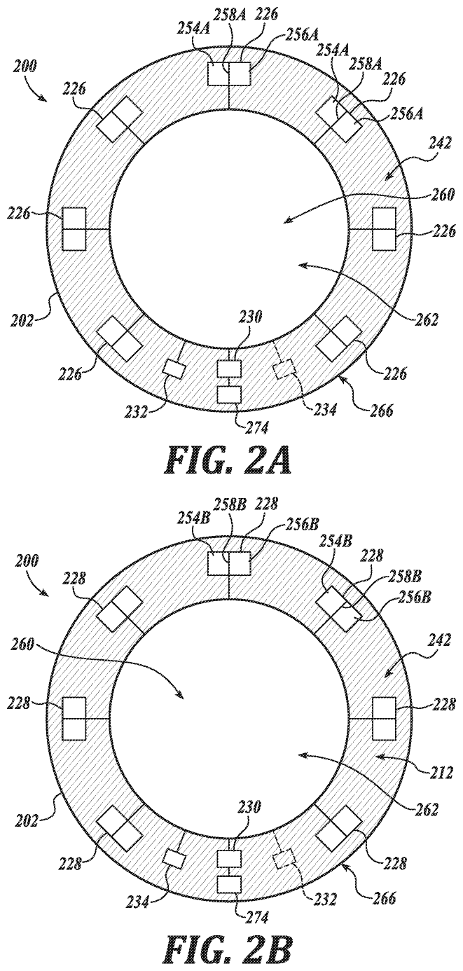

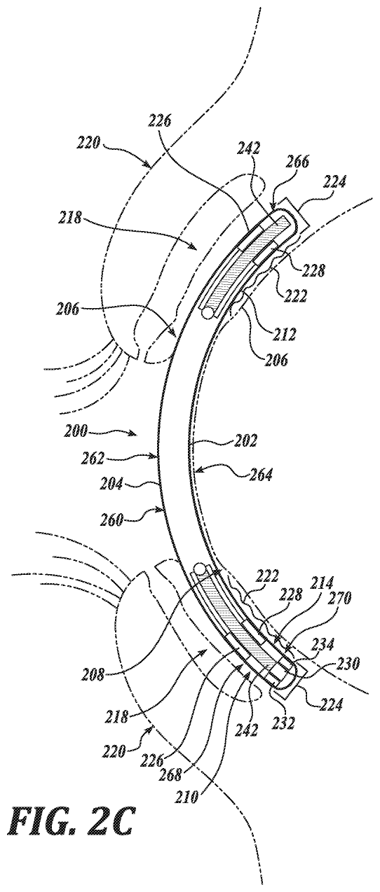

In an embodiment, the ophthalmic devices of the present disclosure include reactants of a chemical reaction configured to heat and/or cool a surface of the ophthalmic devices. In that regard, attention is directed to FIGS. 2A-2C illustrating ophthalmic device 200, in accordance with an embodiment of the present disclosure. FIG. 2A is a top-down plan view of the ophthalmic device 200. FIG. 2B is a bottom-up plan view of the ophthalmic device 200. FIG. 2C is a cross-sectional view of the ophthalmic device 200 shown mounted to a corneal surface of an eye 208, in accordance with an embodiment of the disclosure.

The ophthalmic device 200 includes a housing 202 including a convex anterior surface 204 and a concave posterior surface 212; and a thermal regulator 224 including a heating element 226 positioned to heat the convex anterior surface 204 and a cooling element 228 positioned to cool the concave posterior surface 212. In the illustrated embodiment, the ophthalmic device 200 is a contact lens 200 comprising an optic zone 260 disposed in a central portion 262 of the housing 202 shaped to be mounted to a corneal surface 264 of the eye 208. The optic zone 260 is optically transmissive for transmission of visible light, such that a user can see when the ophthalmic device 200 is mounted to the eye 208. In an embodiment, the optic zone 260 of the ophthalmic device 200 has an optical power, such as to correct for a refractive error of the eye 208.

While a contact lens 200 is shown, it will be understood that the ophthalmic devices can take other forms, as discussed further herein with respect to FIGS. 1A-1C. In that regard, the ophthalmic device 200 including the housing 202 may take the form of an underlid device shaped to be mounted to a conjunctival surface under an eyelid 220 of the eye 208. See, for example, FIG. 1C.

In the illustrated embodiment, the thermal regulator 224 is disposed about a periphery 266 of the housing 202. As shown in FIG. 2C, the thermal regulator 224 including the heating element 226 and the cooling element 228 is configured to contact the conjunctiva 206 of the eye 208 when the contact lens 200 is mounted to the corneal surface 264 of the eye 208.

As above, the thermal regulator 224 includes a heating element 226 configured to heat the convex anterior surface 204. The thermal regulator 224 includes reactants 254A, 256A, 254B, and 256B of chemical reactions disposed within the housing 202. In the illustrated embodiment, the heating element 226 includes a first reactant 254A of a chemical reaction; and a second reactant 256A of the chemical reaction, wherein the chemical reaction is exothermic. In this regard, as the first reactant 254A and second reactant 256A are combined, the chemical reaction proceeds generating heat.

In an embodiment, the first reactant 254A includes iron metal and the second reactant 256A includes oxygen. Further chemical reaction reactants and catalysts can include water, cellulose, vermiculite, activated carbon, and salt.

The thermal regulator 224 further includes a breakable barrier 258A shaped to separate the first reactant 254A and the second reactant 256A. In an embodiment, the breakable barrier 258A includes an electrically conductive layer, such as a thin gold layer, configured to degrade with the application of sufficient electrical current thereby bringing the first reactant 254A in contact with the second reactant 256A. Once the breakable barrier 258A is broken, the chemical reaction proceeds and generates heat, heating the convex anterior surface 204 and Meibomian glands 218 when the ophthalmic device 200 is mounted to the eye 208.

As shown, the breakable barrier 258A is operably coupled to a controller 230 and a power source 274. In an embodiment, the controller 230 includes logic that, when executed by the controller 230, causes the ophthalmic device 200 to perform operations includes applying electrical current to the breakable barrier 258A sufficient to degrade the breakable barrier 258A to place the first reactant 254A and second reactant 256A in contact. In an embodiment, the controller 230 includes logic that, when executed by the controller 230, causes the ophthalmic device 200 to perform operations includes sequentially applying current to each of the breakable barriers 258A in order to sequentially introduce first reactants 254A and second reactants 256A to extend a period of heat treatment. In this regard, the ophthalmic device 200 may be used for an extended treatment period as different breakable barriers 258A are sequentially broken and chemical reactions are allowed to proceed over an extended period of time. After treatment, such as after the chemical reaction has reached equilibrium, the ophthalmic device 200 may be discarded and replaced.

The thermal regulator 224 further includes a cooling element 228 configured to cool the concave posterior surface 212. As shown in FIG. 2B, the cooling element 228 includes a first reactant 254B of a chemical reaction; a second reactant 256B of the chemical reaction; and a breakable barrier 258B shaped to separate the first reactant 254B and the second reactant 256B, wherein the chemical reaction is endothermic. In that regard, as the first reactant 254B and second reactant 256B are combined, such as through breaking the breakable barrier 258B, the concave posterior surface 212 is cooled, suitable to cool scleral nerves 222 of an eye 208 when the ophthalmic device 200 is mounted to the corneal surface 264 of the eye 208. As shown, the thermal regulator 224 the breakable barriers 258B are operatively coupled to the controller 230. As above, breakable barriers 258B may be broken with electrical current supplied by, for example, power source 274 in response to instructions received from controller 230.

In an embodiment, the first reactant 254B is water and the second reactant 256B is selected from the group consisting of ammonium nitrate, calcium ammonium nitrate, urea, and combinations thereof.

In an embodiment, the controller 230 includes logic that, when executed by the controller 230, causes the ophthalmic device 200 to perform operations including applying current to the breakable barrier 258B sufficient to degrade the breakable barrier 258B. In an embodiment, the controller 230 includes logic that, when executed by the controller 230, causes the ophthalmic device 200 to perform operations including sequentially applying current to each of the breakable barriers 258B in order to sequentially introduce first reactants 254B and second reactants 256B to extend a period of cooling treatment.

While electrically conductive breakable barriers 258A and 258B are illustrated, it will be understood that other breakable barriers are possible, such as breakable barriers configured to break when, for example, pressed by hands of a user.

As above, the thermal regulator 224 includes a heating element 226 and a cooling element 228. In the illustrated embodiment, the ophthalmic device 200 further includes a thermal insulator 242 disposed between the heating element 226 and the cooling element 228 to thermally insulate the heating element 226 from the cooling element 228. In this regard, the ophthalmic device 200 is configured to prevent or limit heat transfer between the heating element 226 and the cooling element 228. Such thermal isolation is particularly useful when heating and cooling of the eye 208 are performed simultaneously.

The ophthalmic device 200 is shown to further include a temperature sensor 232 configured to generate a temperature signal based on a temperature of the Meibomian glands 218 and an osmolality sensor 234 configured to generate an osmolality signal based on an osmolality of a tear solution of the eye 208. The temperature sensor 232 and osmolality sensor 234 are operatively coupled to controller 230 and power source 274. As shown in FIG. 2C, when the ophthalmic device 200 is mounted to the corneal surface 264 of the eye 208, the temperature sensor 232 is positioned to contact a portion 210 of the conjunctiva 206, including the palpebral conjunctiva 268, adjacent to the Meibomian glands 218. Likewise, the osmolality sensor 234 is positioned to contact a portion 214 of the conjunctiva 206, including the bulbar conjunctiva 270, adjacent to scleral nerves 222 of the eye 208 when the ophthalmic device 200 is mounted to the corneal surface 264. In this regard, the ophthalmic device 200 may be configured to heat the convex anterior surface 204 with the heating element 226 based upon the temperature signal and cool the concave posterior surface 212 with the cooling element 228 based upon the osmolality signal, as discussed further herein with respect to FIGS. 1A-1C. As discussed further herein with respect to FIGS. 1A-1C, the osmolality sensor 232 may also be configured to generate a moisture signal indicative of an amount of moisture, such as an amount of a tear solution, in contact with the osmolality sensor 232.

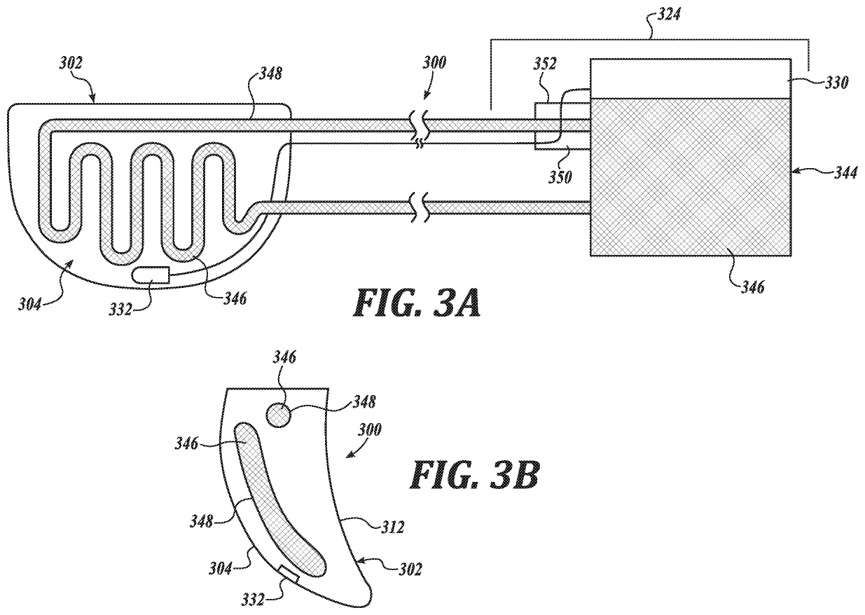

In an embodiment, the ophthalmic devices of the present disclosure include a microchannel for transport of a thermal regulation fluid for convective heating and/or cooling of an eye. In that regard, attention is directed to FIGS. 3A and 3B, illustrating an ophthalmic device 300, in accordance with an embodiment of the disclosure. FIG. 3A is a cross-sectional view of an ophthalmic device 300. FIG. 3B is a cross-sectional view of a portion of the ophthalmic device 300.

The ophthalmic device 300 is shown to include a housing 302 and a thermal regulator 324. As shown in FIG. 3B, the housing 302 includes a convex anterior surface 304 and a concave posterior surface 312. As discussed further herein with respect to FIG. 1C, the convex anterior surface 304 is configured to contact a portion of conjunctiva, such as palpebral conjunctiva, when the housing 302 is mounted under an eyelid of the eye. Likewise, the concave posterior surface 312 is shaped to contact another portion of conjunctiva, such as bulbar conjunctiva, when the ophthalmic device 300 is mounted under the eyelid.

The thermal regulator 324 is shown to include a fluid reservoir 344 carrying a thermal regulation fluid 346, a microchannel 348 disposed in an underlid body of the ophthalmic device 300 in fluid communication with the fluid reservoir 344; a fluid pump 350 configured to flow the thermal regulation fluid 346 through the microchannel 348; and a heat exchanger 352 configured to regulate a temperature of the thermal regulation fluid 346. In operation, the thermal regulation fluid 346 is transported through the microchannel 348, thereby heating or cooling a portion of the housing 302, such as through convection. The thermal regulation fluid 346 can be a fluid configured to transfer heat to the housing 302. In an embodiment, the thermal regulation fluid 346 is selected from the group consisting of water, a saline solution, and ethylene glycol.

A portion of the thermal regulator 324 may be shaped to extend out from under an eyelid when the housing 302 is mounted under the eyelid. In this regard, portions of the thermal regulator 324 including, for example, the fluid reservoir 344, the fluid pump 350, and the heat exchanger 352, may be disposed outside of the housing 302 to be placed on a surface, such as a table, or placed in a pocket during treatment.

In the illustrated embodiment, the microchannel 348 is positioned adjacent to the anterior convex surface 304. As shown, the microchannel 348 has a serpentine arrangement for effecting efficient heat transfer between the thermal regulation fluid 346 and the portion of the eye contacted by the ophthalmic device 300. The heat exchanger 352 includes a heating element 326 configured to heat the thermal regulation fluid 346 to a temperature greater than a temperature of the Meibomian glands. In this regard, as the thermal regulation fluid 346 is transported through the microchannel 348, the ophthalmic device 300 is configured to heat the Meibomian glands when the housing 302 including, for example, the convex anterior surface 304 is mounted under an eyelid of an eye, thereby, for example, inducing expression of meibum from the Meibomian glands.

As shown, the ophthalmic device 300 includes a temperature sensor 332 disposed adjacent to the convex anterior surface 304. Such a temperature sensor 332 is configured to contact a portion of conjunctiva adjacent to Meibomian glands of an eye when the housing 302 of the ophthalmic device 300 is mounted under an eyelid. As discussed further herein with respect to FIGS. 1A-1C, the temperature sensor 332 is configured to generate a temperature signal for receipt by the controller 330 and based on a temperature of the Meibomian glands. In an embodiment, the controller 330 includes logic that, when executed by the controller 330, causes the ophthalmic device 300 to perform operations including heating the convex anterior surface 304 with the heating element 326 to heat the Meibomian glands.

In an embodiment, the thermal regulator 324 may heat the thermal regulation fluid 346 based on the temperature of the Meibomian glands. Likewise, the fluid pump 350 may be configured to pump the thermal regulation fluid 346 at a fluid a flow rate based upon the temperature of the Meibomian glands. In this regard, the ophthalmic device 300 is configured to heat the Meibomian glands to induce expression of meibum therefrom without, for example, overheating the Meibomian glands.

While the illustrated ophthalmic device 300 is configured to heat the Meibomian glands of the eye, the ophthalmic device 300 may be configured to cool a portion of the eye, such as scleral nerves of the eye, such as when mounted under an eyelid of the eye. In that regard, the heat exchanger 352 can include a cooling element (see FIGS. 1A-1C and 2A-2C) configured to cool the thermal regulation fluid 346. As such thermal regulation fluid 346 is pumped through the microchannel 348, a portion of the housing 302 including the concave posterior surface 312 is cooled. In this regard, at least a portion of the microchannel 348 is disposed adjacent to the concave posterior surface 312 of the housing 302, which is positioned adjacent to conjunctiva adjacent to scleral nerves when the ophthalmic device 300 is mounted to an eye. When cooled thermal regulation fluid 346, such as thermal regulation fluid 346 having a temperature less than a temperature of scleral nerves of the eye, is pumped through the microchannel 348 the concave posterior surface 312 is cooled, such as through convection, inducing tear production in the eye.

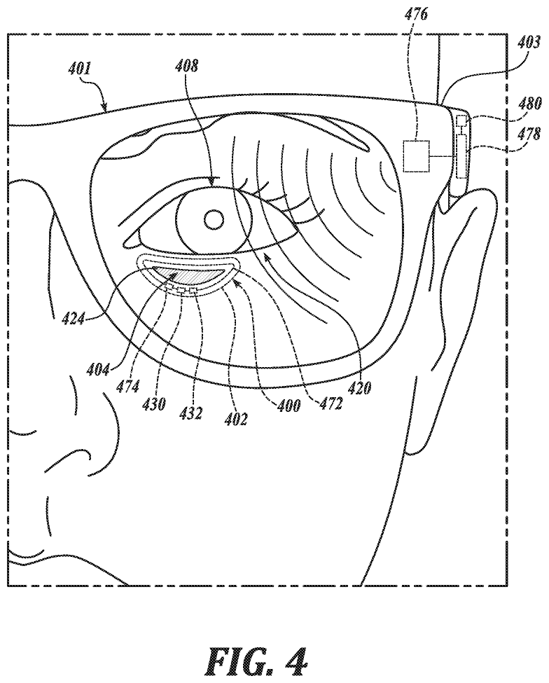

In another aspect, the present disclosure provides a system for treating dry eye, the system including an ophthalmic device for heating and/or cooling a portion of an eye and an auxiliary component configured to wirelessly power the ophthalmic device from a remote position external to the ophthalmic device. FIG. 4 is a perspective view of a system 401 including ophthalmic device 400 and auxiliary component 403, in accordance with an embodiment of the disclosure. The ophthalmic device 400 may be an example of ophthalmic devices 100, 200, and/or 300. In an embodiment, the ophthalmic device 400 is substantially similar to ophthalmic devices 100, 200, and/or 300

The ophthalmic device 400 includes a housing 402 including a convex anterior surface 404 and a concave posterior surface (see, for example, FIGS. 1B and 1C). As discussed further herein, the convex anterior surface 404 is shaped to contact a portion of conjunctiva, such as palpebral conjunctiva, of the eye 408 adjacent to Meibomian glands of the eye 408 when the ophthalmic device 400 is mounted under an eyelid 420 of the eye 408. Correspondingly, the concave posterior surface is shaped to contact a second portion of the conjunctiva adjacent to scleral nerves of the eye 408, such as bulbar conjunctiva, when the ophthalmic device 400 is mounted under the eyelid 420.

The ophthalmic device 400 includes a thermal regulator 424. As discussed further herein, such a thermal regulator 424 includes at least one of a heating element (not shown, see, for example, FIGS. 1A-1C and 2A-2C) positioned to heat the convex anterior surface 404 or a cooling element (not shown, see, for example, FIGS. 1A-1C and 2A-2C) positioned to cool the concave posterior surface. In this regard, the thermal regulator 424 is configured to perform one or more functions including heating the Meibomian glands of an eye and/or cooling scleral nerves of an eye.

Ophthalmic device 400 is shown to include a power source 474 for powering the thermal regulator 424. Ophthalmic device 400 includes controller 430 operatively coupled to power source 474. In an embodiment, controller 430 includes logic that, when executed by the controller 430, causes the ophthalmic device 400 to perform operations including heating the convex anterior surface 404 to heat the Meibomian glands. As shown, the ophthalmic device 430 includes a temperature sensor 432 configured to generate a temperature signal based on a temperature of the Meibomian glands. In this regard, the ophthalmic device 400 is configured to heat the convex anterior surface 404 based upon the temperature signal, as discussed above.

The auxiliary component 403 is configured to wirelessly power the power source 474, such as through inductive coupling. By wirelessly powering the ophthalmic device 400 with the auxiliary component 403, the ophthalmic device 400 may remain mounted to the eye 408 for cooling and/or heating in treatment of dry eye longer than without such wireless power. As shown, the auxiliary component 403 includes a wireless transmitter 476 configured to wirelessly transmit power, such as radio-frequency power, optical power, and the like. The auxiliary component 403 further includes an auxiliary controller 478 operatively coupled to auxiliary power source 480. In an embodiment, auxiliary controller 478 includes logic that, when executed by the auxiliary controller 478, causes the auxiliary component 403 to execute operations including, for example, wirelessly transmitting power from the auxiliary power source 480 with the wireless transmitter 476 for receipt by the wireless receiver 472.

As above, the ophthalmic device 400 includes a wireless receiver 472, such as an inductive coil, configured to receive the power. The ophthalmic device 400 further includes a power source 474 configured to receive the power from the wireless receiver 472.

In an embodiment, the thermal regulator 424 comprises a Peltier heat pump (not shown, see, for example, FIGS. 1A-1C). As discussed further herein with respect to FIGS. 1A-1C, such a thermal regulator 424 includes heating element and the cooling element, wherein the heating element comprises a hot side of the Peltier heat pump positioned to heat the convex anterior surface 404, and wherein the cooling element comprises a cold side of the Peltier heat pump positioned to cool the concave posterior surface. While Peltier heat pumps are sources of heating and cooling, certain Peltier heat pumps heat and cool relatively inefficiently. In this regard, by wirelessly powering the thermal regulator 424 including the Peltier heat pump with the auxiliary component 403, the ophthalmic device 400 can remain mounted to the eye for a longer period of time, such as a period of treatment for dry eye greater than without wireless power.

In order to effect efficient transfer of wireless power between the ophthalmic device 400 and the auxiliary component 403, the auxiliary component 403 may be configured to be stored or worn adjacent to the ophthalmic device 400 when the ophthalmic device 400 is mounted to the eye 408. In the illustrated embodiment, the auxiliary component 403 is a pair of eyeglasses 403. While eyeglasses 403 are shown, it will be understood that the auxiliary component 403 can take other forms configured to be worn close to an eye, such as a necklace, earrings, a hat, a headband, and the like.

In an embodiment, the auxiliary controller 478 is configured to send signals, such as temperature signals, osmolality signals, moisture signals, and the like, indicative of conditions of the eye 408 to a remote location suitable for receipt and analysis by a healthcare provider. Such signals may be sent with, for example, wireless transmitter 476. Likewise, in an embodiment, the auxiliary component 403 is configured to receive signals, such as instructions from the healthcare provider, to alter a course of treatment based upon the signals sent. In an embodiment, such instructions are received with the wireless transmitter 476 where the wireless transmitter 476 is a wireless transceiver 476.

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific embodiments disclosed in the specification. Rather, the scope of the invention is to be determined entirely by the following claims, which are to be construed in accordance with established doctrines of claim interpretation.

What is claimed is:

1. An ophthalmic device comprising:
a housing including a convex anterior surface and a concave posterior surface, wherein the convex anterior surface is shaped to contact a portion of conjunctiva of an eye adjacent to Meibomian glands of the eye when the ophthalmic device is mounted under an eyelid of the eye, and wherein the concave posterior surface is shaped to contact a second portion of the conjunctiva adjacent to scleral nerves of the eye when the ophthalmic device is mounted under the eyelid; and
a thermal regulator disposed in or on the housing, the thermal regulator including at least one of a heating element positioned to heat the convex anterior surface or a cooling element positioned to cool the concave posterior surface when the ophthalmic device is mounted under the eyelid.

2. The ophthalmic device of claim 1, wherein the thermal regulator includes the heating element, the ophthalmic device further comprising:
a controller operatively coupled to the thermal regulator, the controller including logic that, when executed by the controller, causes the ophthalmic device to perform operations including:
heating the convex anterior surface with the heating element to heat the Meibomian glands.

3. The ophthalmic device of claim 2, wherein the thermal regulator includes the cooling element, and wherein the controller includes further logic that, when executed by the controller, causes the ophthalmic device to perform further operations including:
cooling the concave posterior surface with the cooling element to cool the scleral nerves.

4. The ophthalmic device of claim 2, further comprising a temperature sensor configured to generate a temperature signal based on a temperature of the Meibomian glands, wherein the controller is operatively coupled to the temperature sensor, the controller further including logic that, when executed by the controller, causes the ophthalmic device to perform operations including:
heating the convex anterior surface with the heating element based upon the temperature signal.

5. The ophthalmic device of claim 3, further comprising an osmolality sensor configured to generate an osmolality signal based on an osmolality of a tear solution of the eye, wherein the controller is operatively coupled to the osmolality sensor, the controller further including logic that, when executed by the controller, causes the ophthalmic device to perform operations including:
cooling the concave posterior surface with the cooling element based upon the osmolality signal.

6. The ophthalmic device of claim 1, wherein the thermal regulator comprises a Peltier heat pump comprising the heating element and the cooling element, wherein the heating element comprises a hot side of the Peltier heat pump positioned to heat the convex anterior surface, and wherein the cooling element comprises a cold side of the Peltier heat pump positioned to cool the concave posterior surface.

7. The ophthalmic device of claim 1, wherein the thermal regulator comprises the heating element and the cooling element, and wherein the ophthalmic device further comprises a thermal insulator disposed between the heating element and the cooling element to thermally insulate the heating element from the cooling element.

8. The ophthalmic device of claim 1, wherein the thermal regulator comprises:
a fluid reservoir carrying a thermal regulation fluid;
a microchannel disposed in the housing in fluid communication with the fluid reservoir;
a fluid pump configured to flow the thermal regulation fluid through the microchannel; and
a heat exchanger configured to regulate a temperature of the thermal regulation fluid.

9. The ophthalmic device of claim 8, wherein the heat exchanger includes the heating element configured to heat the thermal regulation fluid to a temperature greater than a temperature of the Meibomian glands, and wherein the microchannel is positioned adjacent to the anterior convex surface.

10. The ophthalmic device of claim 8, wherein the heat exchanger includes the cooling element configured to cool the thermal regulation fluid to a temperature less than a temperature of the scleral nerves, and wherein the microchannel is positioned adjacent to the posterior convex surface.

11. The ophthalmic device of claim 1, wherein the thermal regulator comprises:
a first reactant of a chemical reaction;
a second reactant of the chemical reaction; and
a breakable barrier shaped to separate the first reactant and the second reactant.

12. The ophthalmic device of claim 11, wherein the thermal regulator includes the heating element and the chemical reaction is exothermic.

13. The ophthalmic device of claim 11, wherein the thermal regulator includes the cooling element and the chemical reaction is endothermic.

14. The ophthalmic device of claim 1, wherein the ophthalmic device is a contact lens comprising an optic zone disposed in a central portion of the housing shaped to be mounted to a corneal surface of the eye, and wherein the thermal regulator is disposed about a periphery of the housing.

15. The ophthalmic device of claim 1, wherein the portion of conjunctiva adjacent to the Meibomian glands includes the palpebral conjunctiva and the portion of conjunctiva adjacent to the scleral nerves includes the bulbar conjunctiva.

16. A system comprising:
an ophthalmic device including:
a housing including a convex anterior surface and a concave posterior surface, wherein the convex anterior surface is shaped to contact a portion of conjunctiva of an eye adjacent to Meibomian glands of the eye when the ophthalmic device is mounted under an eyelid of the eye, and wherein the concave posterior surface is shaped to contact a second portion of the conjunctiva adjacent to scleral nerves of the eye when the ophthalmic device is mounted under the eyelid;
a thermal regulator disposed in or on the housing, the thermal regulator including at least one of a heating element positioned to heat the convex anterior surface or a cooling element positioned to cool the concave posterior surface when the ophthalmic device is mounted under the eyelid; and
an auxiliary component configured to wirelessly power the thermal regulator from a remote position external to the ophthalmic device.

17. The system of claim 16, wherein the thermal regulator comprises an inductive coil configured to wirelessly receive power from the auxiliary component.

18. The system of claim 16, wherein the auxiliary component includes a wireless transmitter configured to wirelessly transmit power, and wherein the ophthalmic device comprises a wireless receiver configured to receive the power and a power source configured to receive the power from the wireless receiver.

19. The system of claim 16, wherein the thermal regulator comprises a Peltier heat pump comprising the heating element and the cooling element, wherein the heating element comprises a hot side of the Peltier heat pump positioned to heat the convex anterior surface, and wherein the cooling element comprises a cold side of the Peltier heat pump positioned to cool the concave posterior surface.

20. The system of claim 16, wherein the thermal regulator comprises the heating element and the cooling element, and wherein the ophthalmic device further comprises a thermal insulator disposed between the heating element and the cooling element to thermally insulate the heating element from the cooling element.
